(19) United States
(12) Patent Application Publication
(10) Pub. No.: US 2018/0036343 A1
(43) Pub. Date: Feb. 8, 2018

(54) Pain Relief Salt Composition and Method

(71) Applicant: Carolyn Wallace, Leesburg, GA (US)

(72) Inventor: Carolyn Wallace, Leesburg, GA (US)

(21) Appl. No.: 15/637,610

(22) Filed: Jun. 29, 2017

Related U.S. Application Data
(60) Provisional application No. 62/370,784, filed on Aug. 4, 2016.

(51) Int. Cl. A61K 35/08; C11D 7/10; A61K 36/53; A61K 36/752; A61K 45/06; A61K 36/9068

(52) U.S. Cl. CPC A61K 35/08; A61K 45/06; A61K 36/9068; A61K 36/53; A61K 36/752; C11D 7/10

(57) ABSTRACT

A pain relief salt composition. An embodiment of the composition includes a blend of proprietary essential oils mixed with Epsom salt and Dead Sea salt. The proprietary essential oils may include lavender oil, lemongrass oil, rosemary oil, patchouli oil, and ginger root oil. The pain relief salt composition is added to a bath to produce a pain relief salt bath. A salt bath prepared with the pain relief salt composition provides the user with temporary relief from aches and pains associated with arthritis, neuralgia, aching muscles, sprains, gout, rheumatism, migraines, and menstruation, along with conditions such as insomnia, hypertension, inflammation, tension, anxiety, and flatulence.

### CROSS REFERENCE TO RELATED APPLICATIONS

This application claims the benefit of U.S. Provisional Application No. 62/370,784 filed on Aug. 4, 2016. The above identified patent application is herein incorporated by reference in its entirety to provide continuity of disclosure.

### FIELD OF THE INVENTION

The present invention relates to chemical compositions. More specifically, the present invention provides a pain relief salt composition for use in preparing a salt bath that provides pain relief and other therapeutic properties.

### BACKGROUND OF THE INVENTION

People with various debilitating illnesses often suffer from constant pain. Those with arthritis, neuralgia, aching muscles, sprains, stress, gout, and other conditions may seek temporary relief from the pain. Some people may continue to see a decline in their quality of life if these aches and pains are left untreated. People may be less productive at work, be unable to perform normal daily routines, and much more. Often these chronic aches and pains also cause insomnia.

Some methods of relieving pain include taking prescription pain medication. However, this medication is often highly addictive, and often has many undesirable side effects that may be worse than the initial pain itself. Insomnia medication is also often unreliable and highly addictive. Other methods include applying heat or ice to a pained area. However, heat or ice alone is insufficient to relieve chronic pain or particularly intense pain. It is therefore desirable to provide a pain relief salt composition that can be used to prepare a salt bath, wherein the salt bath provides pain relief, moisturizes skin, reduce insomnia and provides additional therapeutic benefits to the bather.

In light of the devices disclosed in the prior art, it is submitted that the present invention substantially diverges in design elements from the prior art and consequently it is clear that there is a need in the art for an improvement to existing pain relief devices and compositions. In this regard the instant invention substantially fulfills these needs.

### SUMMARY OF THE INVENTION

In view of the foregoing disadvantages inherent in the known types of pain relief salt compositions now present in the prior art, the present invention provides a pain relief salt composition wherein the same can be utilized for providing convenience for the user when preparing a salt bath for therapeutic benefits. The pain relief salt composition comprises lavender oil, lemongrass oil, rosemary oil, patchouli oil, ginger root oil, Epsom salt, and Dead Sea salt. The pain relief salt can be dissolved in a warm bath, wherein the oils are aromatized and volatilized such that their therapeutic properties are imparted to the user. The combination of oils allow individuals to treat symptoms of arthritis, insomnia, inflammation, and anxiety, as well as symptoms caused by a variety of other ailments.

One object of the present invention is to provide a pain relief salt composition that can be used to prepare a salt bath for relieving pain and providing other therapeutic benefits.

Another object of the present invention is to provide a pain relief salt composition having a pleasant aromatic quality.

A further object of the present invention is to provide a pain relief salt composition that includes only natural ingredients.

Other objects, features and advantages of the present invention will become apparent from the following detailed description.

### DETAILED DESCRIPTION OF THE INVENTION

Although the characteristic features of this invention will be particularly pointed out in the claims, the invention itself and manner in which it may be made and used may be better understood after a review of the following detailed description.

For the purposes of presenting a brief and clear description of the present invention, the preferred embodiment will be discussed as used for preparing a salt bath for relieving pain and other therapeutic purposes. The following detailed description is intended for representative purposes only and should not be considered to be limiting in any respect.

The present invention provides a composition for pain relief salts. In one embodiment of the invention, the composition generally includes a proprietary blend of oils mixed with Epsom salt and Dead Sea salt. The coarseness of the Dead Sea salt can vary from fine grain to coarse grain. Some embodiments may utilize a fine grain, while some utilize a coarse grain, and others utilize a mixture of fine and coarse grains. The grain size determines the exfoliating properties of the pain relief salt bath prepared using the pain relief salt composition of the present invention.

The proprietary oils provide aromatic and therapeutic properties to the pain relief salt composition. In one embodiment, the proprietary blend of oils includes lavender oil, lemongrass oil, rosemary oil, patchouli oil, and ginger root oil. The combination essential oils provide aromatic and therapeutic properties to the pain relief bath salts.

In an exemplary embodiment, the pain relief salt composition comprises 0.4% to 2.5% by weight of lavender oil, 0.4% to 2.5% percent by weight of lemongrass oil, 0.4% to 2.5% percent by weight of rosemary oil, 0.4% to 2.5% percent by weight of patchouli oil, 0.1% to 1.5% by weight of ginger root oil, 20% to 30% by weight of Epsom salt, and 70% to 80% by weight of Dead sea salt. This specific combination of ingredients is configured to provide the maximum amount of aromatic and therapeutic properties.

In order to compose the pain relief bath salts, measured amounts Epsom salts and Dead Sea salts are combined in a bowl and stirred until no lumps are visible. The measured amounts of essential oils are then poured into a spray bottle. The essential oils are applied to the salts as a light mist from the spray bottle. After the essential oil mixture is added, the composition is stirred, and additional oil mixture is sprayed, alternating between misting and stirring until the entire amount of oil is utilized. The composition is then packaged in a suitable jar or other container.

In order to prepare a bath with the pain relief salts, an individual may add half a cup of the pain relief salt composition to a warm bath just prior to entering, adjusting the amount according to personal preference. The amount of pain relief salt composition may also vary according to the size of the bathtub and the amount of water in the bathtub. The individual may then soak, relax, and inhale the vapors provided by the proprietary blend of essential oils for as long as desired, but not less than fifteen minutes. The essential oils interact with the Epsom and Dead Sea salts when the pain relief salt composition is dissolved in warm water, allowing the individual to obtain temporary relief from aches and pains associated with arthritis, neuralgia, aching muscles, sprains, gout, rheumatism, migraines, and menstruation, along with conditions such as insomnia, hypertension, inflammation, tension, anxiety, and flatulence.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A pain relief salt composition, comprising:
lavender oil;
lemongrass oil;
rosemary oil;
patchouli oil;
ginger root oil;
Epsom salt;
Dead Sea salt.

2) The pain relief salt composition of claim 1, wherein the amount of the lavender oil in the composition is within a range of 0.4% to 2.5% by weight.

3) The pain relief salt composition of claim 1, wherein the amount of the lemongrass oil in the composition is within a range of 0.4% to 2.5% by weight.

4) The pain relief salt composition of claim 1, wherein the amount of the rosemary oil in the composition is within a range of 0.4% to 2.5% by weight.

5) The pain relief salt composition of claim 1, wherein the amount of the patchouli oil in the composition is within a range of 0.4% to 2.5% by weight.

6) The pain relief salt composition of claim 1, wherein the amount of the ginger root oil in the composition is within a range of 0.1% to 1.5% by weight.

7) The pain relief salt composition of claim 1, wherein the amount of the Epsom salt in the composition is within a range of 20% to 30% by weight.

8) The pain relief salt composition of claim 1, wherein the amount of the Dead Sea salt in the composition is within a range of 70% to 80% by weight.

9) A pain relief salt composition consisting essentially of:
0.4% to 2.5% by weight of lavender oil;
0.4% to 2.5% percent by weight of lemongrass oil;
0.4% to 2.5% percent by weight of rosemary oil;
0.4% to 2.5% percent by weight of patchouli oil;
0.1% to 1.5% by weight of ginger root oil;
20% to 30% by weight of Epsom salt;
70% to 80% by weight of Dead sea salt.
